(19) United States
(12) Patent Application Publication
(10) Pub. No.: US 2015/0030565 A1
(43) Pub. Date: Jan. 29, 2015

(54) PURIFICATION OF FLAVIVIRUSES

(71) Applicant: Sanofi Pasteur Biologics, LLC, Cambridge, MA (US)

(72) Inventors: Sophia Mundle, Boston, MA (US); Stephen Anderson, Arlington, MA (US)

(73) Assignee: Sanofi Pasteur Biologics, LLC, Cambridge, MA (US)

(21) Appl. No.: 14/369,796

(22) PCT Filed: Jan. 8, 2013
(86) PCT No.: PCT/US2013/020686

§ 371 Date: Jun. 30, 2014

Related U.S. Application Data
(60) Provisional application No. 61/584,461, filed on Jan. 9, 2012.

(51) Int. Cl. C12N 7/00

(52) U.S. Cl. CPC C12N 7/00; C12N 2770/24133; C12N 2770/24134; C12N 2770/24151
USPC 424/93.2; 435/239; 424/93.6

(57) ABSTRACT

The present invention provides a method to prepare purified enveloped (e.g., flavivirus) viral particle preparations employing ion exchange chromatography and tangential flow filtration.

### CROSS-REFERENCE TO RELATED APPLICATIONS

This application claims benefit of U.S. Ser. No. 61/584,461 filed Jan. 9, 2012, the contents of which are incorporated herein by reference.

### FIELD OF THE INVENTION

The present invention relates to methods and compositions for purification of enveloped viruses. More specifically, the present invention relates to methods and compositions for purification of flaviviruses and flavivirus related viral particles, vectors, and other constructs, and compositions. The compositions of the present invention are useful in therapeutic and/or prophylactic medicinal applications in mammals.

### BACKGROUND OF THE INVENTION

The flavivirus genus consists of about 80 enveloped positive strand RNA viruses, many of which are known to cause disease in animals and humans. A number of these flaviviruses use arthropods (e.g., biting ticks and/or mosquitoes) as a means for transmission to virus recipients. Such arthropod-borne viruses (i.e., arboviruses) constitute a major worldwide health concern due to their highly pathogenic nature in humans. (Fernandez-Garcia M D, et al., Cell Host Microbe, 2009, 5:318-328). More specifically, important human arbovirus pathogens include yellow fever (YF), Japanese encephalitis (JE), dengue types 1-4, West Nile (WN) and tick-borne encephalitis (TBE) viruses that exist in nature in life cycles which involve mosquito or tick vectors and avian and/or mammalian competent reservoir hosts. (Gubler D, et al., In: Fields Virology. Edited by Knipe D M, Howley P M, Griffin P E, et al. 5th ed. Philadelphia: Wolters Kluwer, Lippencott Williams and Wilkins; 2007. pp. 1153-1252). The viruses themselves consist of a nucleocapsid containing the capsid protein (C) and the approximately 11 kb positive strand viral RNA genome, which is encased in a lipid envelope containing the major antigenic determinant (the envelope (E) glycoprotein) and membrane (M) protein, which is produced from the prM precursor protein during viral maturation. (Lindenbach B D, et al., In: Fields Virology. Edited by Knipe D M, Howley P M, Griffin P E, et al. Philadelphia: Wolters Kluwer, Lippencott Williams and Wilkins; 2007. pp. 1101-1152). Structurally, the flavivirus virion undergoes a major rearrangement of the surface (E and prM/M) proteins during the maturation process. (Kuhn R J, et al., Cell, 2002, 108:717-725; and Mukhopadhyay S, et al., Nat Rev Microbiol, 2005, 3:13-22).

There has been substantial interest in developing vaccines against flavivirus caused diseases. A number of licensed vaccines have been in wide use to prevent YF, JE, and TBE. These include whole-inactivated vaccines against JE (JE-VAX®, Sanofi Pasteur, Lyon, France, and IXIARO®, Novartis, Basel, Switzerland), and TBE (FSME-IMMUN®, Baxter, Deerfield, Ill., and ENCEPUR® Novartis), and YF (e.g., YF-VAX®, YF-17D-204, Sanofi Pasteur). At present, there are no licensed human vaccines against dengue or WN (Pugachev K V, et al., In: New Generation Vaccines. Edited by Levine M M, Dougan G, Good M F, et al. 4th ed. New York: Informa Healthcare USA; 2010. pp. 557-569) although a number of animal vaccines against WN do exist. (Dauphin G, and Zientara S., Vaccine, 2007, 25:5563-5576; and Pugachev K V, et al., Curr Opin Infect Dis, 2005, 18:387-394). Additionally, subunit, DNA and chimeric-flavivirus approaches have been explored with varying degrees of success. (Coller B A, et al., Drugs, 2010, 13:880-884; and Pulmanausahakul R, et al., African J. of Biotechnology, 2010, 9:7).

In recent years, Sanofi Pasteur has developed a replication-competent, rationally-attenuated chimeric flavivirus vaccine platform (CHIMERIVAX®) seeking to address the need for a new generation of flavivirus vaccines. (Pugachev K V, et al., pp. 557-569; Pugachev K V, et al., pp. 387-394; Arroyo J, et al., J Virol, 2004, 78:12497-12507; Guy B, et al., Vaccine, 2010, 28:632-649; and Rumyantsev A A, et al., Virology, 2010, 396:329-338). These chimeric flaviviruses include capsid and non-structural sequences of a yellow fever virus and pre-membrane and envelope sequences of a second, different flavivirus. CHIMERIVAX®-JE is currently licensed as IMOJEV™, CHIMERIVAX®-dengue is currently in late phase clinical development and CHIMERIVAX®-WN has been pre-clinically evaluated.

Additionally, Sanofi Pasteur is in development of the REPLIVAX® approach, a platform based on highly attenuated flavivirus vectors which has the potential for developing novel recombinant vaccines against numerous targets, flavivirus and non-flavivirus. REPLIVAX® refers to a flavivirus which has been rendered replication defective by a large in-frame deletion of C and/or the prM-E genes. Constructs are propagated in complementing helper cell lines as a single-component pseudoinfectious virus (PIV), or as two-component vaccines in regular naïve cells, where two sub-genomic replicons self-compliment each other. REPLIVAX® constructs undergo a single round of infection and replication in normal cells, e.g., in vivo upon vaccination. Various REPLIVAX® prototypes generated at The University of Texas Medical Branch (Galveston, Tex.) have been described (Mason P W, et al., Virology, 2006, 351:432-443; Shustov A V, et al., J Virol, 2007, 81:11737-11748; Ishikawa T, et al., Vaccine, 2008, 26:2772-2781; Suzuki R, et al., J Virol, 2008, 82:6942-6951; Widman D G, et al., Adv Virus Res, 2008, 72:77-126; Widman D G, et al., Vaccine, 2008, 26:2762-2771; Suzuki R, et al., J Virol, 2009, 83:1870-1880; Widman D G, et al., Vaccine, 2009, 27:5550-5553; Widman D G, et al., Am J Trop Med Hyg, 2010, 82:1160-1167; lshikawa T, et al., Vaccine, 2011, 29:7444-7455; Winkelmann E R, et al., Virology, 2011, 421:96-104; and Winkelmann E R, et al., Vaccine, 2012, 30:1465-1475) and further extensively characterized at Sanofi Pasteur against live attenuated and inactivated vaccine controls demonstrating excellent potential of the approach (Rumyantsev A A, et al., Vaccine, 2011, 29:5184-5194). Unlike CHIMERIVAX® (Rumyantsev A A, et al., pp. 329-338), REPLI VAX® can also be used for delivery of large foreign, non-flavivirus antigens inserted in place of the deleted gene(s) (Rumyantsev A A, et al., pp. 5184-5194).

Clearly, as initially stated above, the interest in flavivirus based delivery systems for various therapeutics and vaccines as well as for prevention of the flavivirus caused diseases themselves remains a high priority in both animal and human medicine. Vaccine development however is often a difficult scientific and developmental path to follow. For instance, in order for a candidate vaccine to pass from the pre-clinical stage to the clinical stage, appropriate upstream and downstream processing steps must be determined for the generation of a pure product. This situation exemplifies one of the obstacles to successful vaccine development.

The field has tried to address this particular development obstacle in a number of ways. Traditional laboratory-scale purification processes for vaccine strain viruses often involve non-scalable, laborious procedures including ultracentrifugation. Modern processes include chromatographic separation of viruses from contaminants and concentration/purification by tangential flow filtration (TFF), ultrafiltration, and diafiltration (UF/DF). Purification of the enveloped viruses poses a particular challenge as they are highly susceptible to shear force generated by normal liquid flow in non-convective (bead-based) systems. More particularly, a number of chromatography-based methods for the purification of flaviviruses have been published. (See, Gresikova M, et al., Acta Virol, 1984, 28:141-143; Crooks A J, et al., J Chromatogr, 1990, 502:59-68; Hermida Diaz C, et al., Rev Cubana Med Trop 1992, 44:171-176; Sugawara K, et al., Biologicals, 2002, 30:303-314; WO2006/122964 (incorporated herein by reference in its entirety); and Ohtaki N, et al., J Virol Methods, 2011, 174:131-135), though all differ significantly from the methods described herein. Previously described methods have been based either on affinity or size separation of the flavivirus virions of interest. To realize the full potential of flavivirus based vector delivery systems and flavivirus mediated disease prevention and/or amelioration new methods of enveloped virus particle, and flavivirus particle, purification are needed that overcome the shortcomings of existing purification approaches.

### SUMMARY OF THE INVENTION

The present invention provides purification procedures for enveloped viral particles. In some embodiments, these enveloped vial particles are useful as therapeutic and/or prophylactic agents against infection and/or disease in mammals. In this respect, the present invention contemplates purification schemes for medicinal agents and/or vaccines useful in human medicine practiced in various age groups (e.g., infants, toddlers, adolescents, adults, and/or the elderly) as well as veterinary medicine as used in production animals and companion animals (e.g., cows, pigs, chickens, sheep, etc., and dogs, cats, horses, etc.).

Some embodiments of the present invention provide methods for purification of infectious flavivirus particles and/or virus like particles (VLPs) based upon both the size and the anionic surface charge of the particle. The particles produced by these methods are not only functional but are also nearly homogenous as compared to particles and/or VLPs prepared by traditional centrifugal concentration methods.

The present invention provides methods to prepare purified enveloped viral particle preparations employing ion exchange chromatography and tangential flow filtration. In particular, preferred embodiments of the present invention provide purification schemes for therapeutic and/or vaccine candidates based on the flavivirus related REPLIVAX®. This technology is based on replication defective (single-cycle) flavivirus variants. In other embodiments, the purification schemes are used for CHIMERIVAX® viruses, which are chimeric flaviviruses including capsid and non-structural sequences of a yellow fever virus and pre-membrane and envelope sequences of a second, different flavivirus (e.g., a West Nile virus, a Japanese encephalitis virus, a dengue virus, or any other flavivirus, such as another flavivirus described herein). The hollow-fiber TFF and convective-flow anion-exchange chromatography-based purification scheme described herein results in about 50-, 60-, 70-, 80-, or 90- (or greater) % recovery of infectious virus titer and can be used to prepare nearly homogenous, highly purified vaccine viruses with titers as high as 1×10⁶, 1×10⁷, 1×10⁸, or 1×10⁹ (or greater) focus forming units (FFU) per mL.

The present invention further provides a method for the purification of flavivirus viral particle from a host (e.g., mammalian) cell culture comprising the steps of:

a. recovering from a host cell culture flavivirus viral particles from the host cells;

subjecting the solution obtained from step (a) to tangential flow filtration;

c. applying the retentate from the tangential flow filtration step to an anion exchange chromatography resin;

d. eluting the flavivirus viral particles from the anion exchange chromatography resin (column); and

e. recovering the purified flavivirus viral particles.

The invention further provides a method wherein before applying the solution obtained from step (a) to tangential flow filtration step the solution obtained from step (a) is treated with an endonuclease to degrade residual host cell DNA.

The present invention also contemplates, in certain embodiments, that the flavivirus viral particles are recovered following one or more physical or chemical procedures to disrupt or lyse host cells. Host cells may be lysed by any number of applicable techniques including, but not limited to, enzymatic means (e.g., lysozyme, lysostaphin, zymolase, cellulase, mutanolysin, glycanases, proteases, mannose, and the like), physical means (e.g., bead method, sonication, high-shear mechanical methods, and the like), liquid N₂, detergents, and/or solvents and the like.

A number of host cell types are contemplated by the present invention, nevertheless, mammalian host cells are preferred.

The present invention further provides a method for the purification of an flavivirus viral particle from a host (e.g., mammalian) cell culture comprising the steps of:

a. treating the host cell culture with a viral releasing agent to release the flavivirus viral particles from the host cells;

b. subjecting the solution obtained from step (a) to tangential flow filtration;

c. applying the retentate from the tangential flow filtration step to an anion exchange chromatography resin;

d. eluting the flavivirus viral particles from the anion exchange chromatography resin (column); and

e. recovering the purified flavivirus viral particles.

The present invention provides a method for the purification of a flavivirus viral particles from a host (e.g., mammalian) cell culture comprising the steps of:

a. recovering from a host cell culture flavivirus viral particles from the host;

b. applying the solution obtained from step (a) to an anion exchange chromatography resin;

c. eluting the flavivirus viral particles from the anion exchange chromatography resin (column);

d. subjecting the eluent from step (c) to tangential flow filtration, and

e. recovering the purified flavivirus viral particles.

In still further embodiments, the present invention provides a pharmaceutically acceptable dosage form of a flavivirus virus (or flavivirus viral particles/PIVs) produced in a host cell culture said flavivirus virus isolated by the method comprising the steps of:

a. recovering from a host cell culture flavivirus viral particles from the host cells;

b. subjecting the solution obtained from step (a) to tangential flow filtration;

c. applying the retentate from the tangential flow filtration step to an anion exchange chromatography resin;

d. eluting the flavivirus viral particles from the anion exchange chromatography resin (column);

e. recovering the purified flavivirus viral particles; and

f. suspending the purified flavivirus viral particles in a pharmaceutically acceptable carrier.

The invention further provides methods wherein before applying the solution obtained from step (a) to the anion exchange chromatography resin the solution obtained from step (a) is treated with an endonuclease to degrade residual host cell DNA.

Additional embodiments further provide methods comprising the step(s) of clarifying the product material (e.g., solution obtained from step (a) by depth filtration prior to (or following) anion exchange chromatography.

Additional embodiments further provide methods comprising the step(s) of clarifying the product material (e.g., solution obtained from step (a) by dead-end filtration prior to (or following) anion exchange chromatography.

The invention further provides the foregoing procedures with an additional step of to concentrate purified viral particles by diafiltration to prepare a solution containing greater than from about 1×10⁶, 1×10⁷, 1×10⁸, to 1×10⁹ PFU/mL.

Further, the invention provides methods of inducing immune response to a flavivirus (or other) antigen by administration of a composition as described herein, as well as use of the compositions described herein in inducing an immune response. These methods can be used to protect against or treat infection by, for example, a flavivirus corresponding to the source of envelope protein of a flavivirus as described herein.

### BRIEF DESCRIPTION OF THE FIGURES

FIGS. 1A-1C show a representative example of the effectiveness of hollow fiber tangential flow filtration of REPLIVAX® PIVs as examined by gel electrophoresis and Western blot. Medium was harvested from infected BHK packaging cells grown in T-225 flasks and clarified (Load). The REPLIVAX® containing cell culture supernatant was then concentrated 2-6-fold by volume to less than 50 mL and diafiltered against 5×50 mL of Buffer A (Permeate). The final TFF product is designated Retentate. RVWNΔC (1A and 1B) and CRVWNΔprM-E/RSV F (1C) constructs were used. Hollow fiber modules with either 100- (1A) or 500-kDa (1B and 1C) MWCO were tested.

FIG. 2 shows the a representative chromatographic elution profile during laboratory scale (0.7 mL CIM® Q disk) bind-and-elute purification of RVWNΔC PIV by monolithic anion exchange. The solid line represents absorbance at 280 nm. The dotted line represents the concentration of salt as a percentage of the high salt buffer (Buffer B, 2 M NaCl). During the sample loading phase (0-35 mL) RVWNΔC particles bind the solid support, while unbound impurities (and some breakthrough PIVs) pass through the column and are collected as the flow through fraction. Elution of bound PIV is achieved by applying a 900 mM NaCl (35% Buffer B) step maintained over about 15 column volumes (40-50 mL). Bound impurities are eluted from the column by step-wise increase of the salt concentration to 2 M NaCl (100% Buffer B) which is maintained over about 30 CV (50-70 mL).

FIG. 3 shows a representative example of viral infectivity of samples throughout the REPLIVAX® purification process. Titers of RVWNΔC and RVWNΔprM-E/RSV F in purification samples are presented as FFU/mL. The clarified cell culture supernatant (Start) and TFF retentate (Retentate) are followed by chromatography fractions. The first three fractions represent the flow through (FT), fractions 4-8 represent the step 1 elution fractions (E1) and fractions 9-14 represent the step 2 elution fractions (E2).

FIG. 4 shows a representative example of a comparison of purity of RVWNΔC and RVWNΔprM-E/RSV F PIVs prepared by CENTRICON® centrifugal concentration (C) versus chromatographic purification (P). SDS-PAGE (CBB) and Western blot (α-WN or α-RSV-F) analysis reveals that the material which was prepared by centrifugal concentration contains a large amount of protein contaminants, in contrast to virus purified by TFF/chromatography. In both cases, the same number of FFU were loaded per lane (4×10⁶ for RVWNΔC and 1×10⁶ for RVWNΔprM-E/RSV F). In the case of the RVWNΔprM-E/RSV F preparation, the purified material contains no free F protein whereas the material after concentration does.

FIG. 5 shows a representative high level example of a flow diagram of the new purification process (right panel) and comparison of yields of infectious REPLIVAX® PIV at different steps during the purification procedure versus CENTRICON® centrifugal concentration (left panel). While the total virus yields are similar, the purification methods (right panel) provided for recovery of high purity virus (e.g., REPLIVAX®).

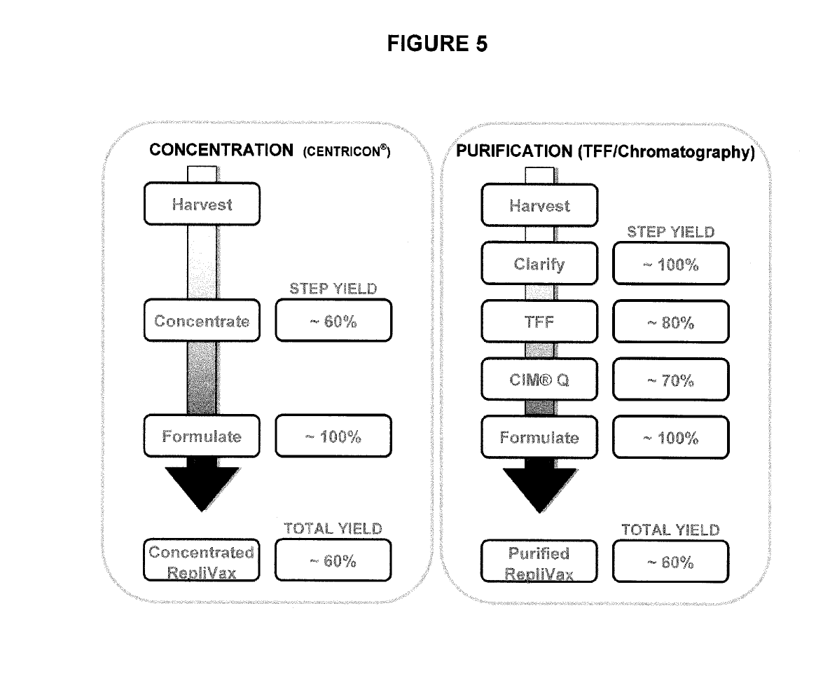

FIG. 6 shows a representative flow diagram of an exemplary flavivirus (e.g., REPLIVAX) purification methodology.

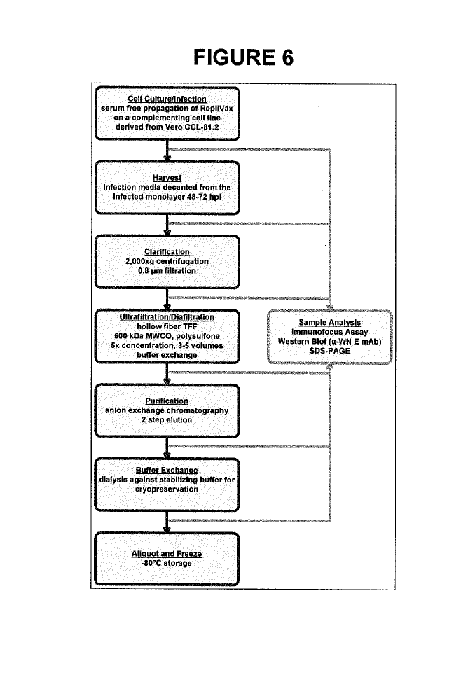

### DESCRIPTION OF THE INVENTION

Examples of enveloped viruses that may be prepared in accordance with the practice of the present invention include, but are not limited to, the following viruses: poxviruses, orthomyxoviruses, paramyxoviruses, and flaviviruses.

A range of virus particles derived from enveloped viruses have been described as useful in the development of vaccines and may be prepared in accordance with the practice of the present invention. The term enveloped viral particle herein is used to collectively refer to wild-type infectious virions, infectious virions containing recombinantly modified genomes, replication competent attenuated infectious virions, as well as non-infectious virus-like particles (VLPs) derived from enveloped viruses

An enveloped virus refers to a viral particle that has an outer wrapping or envelope derived from the infected host cell in a budding process whereby the newly formed virus particles become wrapped in an outer coat that is made from a small piece of the cell's plasma membrane. The budding process by which the virus acquires its envelope results in the viral particles being expelled from the host cells used to grow the virus. Conventionally, some enveloped viruses remain associated with the producer cells. There is a range of time after infection of the host cells where the maximum virus can be released from the cells. The timing of release varies depending on the temperature of infection, the infection media used, the virus which was used to infect the cells, the container in which the cells were grown and infected and the cells themselves.

Identification of this optimal harvest time is readily determined by sampling of the cell culture regularly over the conventional incubation period for the particular enveloped virus to determine the optimal yield.

Examples of enveloped orthomyxoviruses suitable for purification using the methods of the present invention include, but are not limited to, the influenza type A viruses including but not limited to the strains H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7.

Examples of enveloped paramyxovirus suitable for purification using the methods of the present invention include, but are not limited to, human respiratory syncytial virus, measles virus, and mumps virus.

Additional enveloped viruses suitable for purification using the methods of the present invention include, but are not limited to, viruses of the genus flavivirus consisting of about 80 enveloped positive-strand RNA viruses such as West Nile (WN) virus, Japanese Encephalitis virus (JEV), Dengue fever virus, and yellow fever virus (YF). Particular flaviviruses that may be purified in accordance with the present invention include CHIMERIVAX® (Chambers, et al., U.S. Pat. No. 6,696,281 issued Feb. 24, 2004; Guikahoo, U.S. Patent Application Publication Number US 2004/0259224 A1 published Dec. 23, 2004, the entire teachings of which are herein incorporated by reference) and REPLIVAX® (Mason, et al., U.S. Patent Application Publication Number US 2009/0155301 A1 published Jun. 18, 2009; Pugachev, et al., U.S. Patent Application Publication Number US 2010/0184832 A1 published Jul. 22, 2010; and Widman, et al (2008) Adv Virus Res. 72:77-126, the entire teachings of which are herein incorporated by reference) recombinant flaviviruses, and the YF-17D attenuated yellow-fever virus.

In some embodiments, flaviviruses and/or arboviruses suitable for purification using the methods of the present invention include, but are not limited to, the following viral species and type members, Dengue fever, Japanese encephalitis, Kyasanur Forest disease, Murray Valley encephalitis, St. Louis encephalitis, Tick-borne encephalitis, West Nile encephalitis, Yellow fever, Central European encephalitis (TBE-W), Far Eastern encephalitis (TBE-FE), Kunjin, Tyuleniy, Ntaya, Uganda S, Modoc, BVDV (e.g., strains NADL, and 890), CSFV Alfort/187, BDV BD31, and/or GB virus-A, -B, and/or -C. The Hepatitis C viruses closely resemble flaviviruses as well and are contemplated for purification herein using particular embodiments.

In certain embodiments, the newly produced viral particles may be recovered from a cellular supernatant or growth medium. Routine concentration and/or separation techniques can be utilized to enrich for or differentiate the particles of interest from other molecules in the medium prior to (or after) employing the purification methods of the present invention.

In certain other embodiments, rather than harvest the entire cell culture and potentially lysing the host cells and attempting to isolate the newly produced viral particles from the complex cell milieu, it is preferred that the newly formed viral particles be isolated from the surface of the intact host cells. This can be accomplished by simply decanting the medium from the host cells or by exposure of the host cells to a viral releasing agent in some embodiments. Such viral releasing agent is any agent that is capable of disruption of the interaction between the viral particle and the cell surface. In the practice of the present invention, the viral particles are dislodged from the cell surface for instance with solutions containing dextran sulfate, serum free media or phosphate buffered saline. In one embodiment, the viral releasing agent is a solution of the following components: 50 mM potassium glutamate, 10 mM histidine, 0.16 M sodium chloride, 100 μg/mL dextran sulfate MW 6-8 kDa, 10% sucrose, pH 7.5). It was determined experimentally, in some embodiments, that exposure of the cell culture to this viral releasing agent for 24 hours was desirable. Lesser times produced significantly lower yields. Based on experimentation, it is desirable that the culture be exposed to the releasing agent for at least 3 hours, at least 5 hours, at least 8 hours, or between 20 and 24 hours. Optimally, the culture should be exposed to the releasing agent for 24 hours to maximize the yield of viral particles. Viral releasing agents should not be necessary in liberating/obtaining flavivirus particles from host cell cultures as a precursor step to the present purification methods.

In still other embodiments, when performing a depth filtration procedure prior to (or after) anion-exchange chromatography, endonuclease (e.g. Benzonase®) treatment of the viral preparation can improve the efficiency of the process by minimizing fouling of the depth filtration matrix.

As is understood in the art, depth filtration refers to the use of a porous filter medium to clarify solutions containing significant quantities of large particles (e.g., intact cells or cellular debris) in comparison to membrane filtration which would rapidly become clogged under such conditions. A variety of depth filtration media of varying pore sizes are commercially available from a variety of manufacturers such as Millipore, Pall, General Electric, and Sartorius. In the practice of the invention as exemplified herein, SARTO-SCALE® disposable SARTOPURE® PP2, 0.65 μm depth filters (Sartorius Stedim, Goettingen, Germany) were used in certain embodiments. Use of this system resulted in no appreciable loss of virus titer. Incorporation of depth filtration techniques (one or more steps) may be particularly advantageous in the purification of flavivirus particles (e.g., CHIMERIVAX® and/or REPLIVAX® PIVs or vectors) for scaled-up operations.

In other embodiments, the use of dead-end filtration is preferred, particularly, in embodiments optimized for purification of REPLIVAX® PIVs and related vectors.

The principles of anion exchange chromatography are well known in the art, but briefly this method relies on the charge-charge interactions between the particles to be isolated and the charge on the resin used. Since most viruses are negatively charged at physiological pH ranges, the column contains immobilized positively charged moieties. Generally these are quaternary amino groups (Q resins) or diethylaminoethane groups (DEAE resin). In the purification of large particles such as viruses, it has been demonstrated that monolithic supports (e.g., columns) with large (e.g., >1 micron) pore sizes that enable purification of macromolecules such as viruses are advantageous in certain embodiments. The use of such monolithic supports is therefore preferred. Examples of commercially available Q and DEAE resin monolithic supports include the CIM® QA and CIM® DEAE disc (BIA Separations, Villach, Austria). Other anion exchange resins useful in the practice of the present invention include the MUSTANG® Q (Pall, Corp., Port Washington, N.Y.) and the FRACTOGEL® TMAE (Merck, Whitehouse Station, N.J.) resins.

The present invention provides methods and processes for efficiently purifying flavivirus particles such as REPLIVAX® PIVs in a two-step purification method involving hollow-fiber TFF and chromatographic separation using anion exchange monolithic column(s) such as, but not limited to, Convective Interactive Media® (e.g., CIM® Q, BIA Separations, Villach, Austria) without loss of infectivity of the PIVs. Beneficial features of the present methods include, but are not limited to, extremely fast separation (typically the chromatography step takes about 20 min) with high flow rate and low backpressure, high flow-independent binding capacity, high resolution and recovery, and/or simplified handling. CIM® anion exchange monolithic columns are generally described in U.S. Pat. Nos. 4,889,632; 4,923,610; 4,952,349; 5,972,218; 6,319,401; 6,736,973; and 6,664,305 each of which is incorporated by reference herein in its entirety.

The hollow-fiber TFF and chromatographic separation media(s)/cassette(s) useful in the methods of the present invention include polysulfone TFF cassettes of about 55 cm², but TFF membranes can also comprise polyethersulfone, modified polyethersulfone or mixed cellulose ester. The sizes of useful hollow fiber modules vary from micro (volumes of 1-100 mL, 5-20 cm²), to midi (100 mL-3 L, 22-145 cm²), to mini (5-15 L, 1570-10000 cm²), to KrosFlo (10-100 L, 0.785-5.10 m²) modules. CIM® monoliths (supports/columns) are available in volumes of 0.34 mL disc (can be stacked up to four in one housing for 0.34-1.36 mL), or 8, 80, 800 or 8,000 mL columns. The sizes and follow volumes of the various filters, columns, and separation devices, specified herein are exemplary and specific only to certain exemplary embodiments. It is understood that various embodiments of the purification techniques of the present invention can be optimized to make use of one or more TFF separation media(s)/cassette(s) and/or one or more anion exchange chromatography resin(s) placed serially or in parallel in the purification scheme.

While the methods of the present invention are not limited to any mechanism or particular mode or order of operation monolithic chromatographic supports (e.g., CIM® Q) are particularly preferred for use in the disclosed methods for one or more of the following reasons: preferred architecture of the chromatography media, considerations related to mass transport within a monolith and void, and advantageous flow distribution within the column. Furthermore, monolithic chromatographic supports are considered to be an advantageous means for purification of viruses such as flaviviruses and potentially other large biomolecules which may be either limited by diffusion or affected by fluid friction.

In addition to the physicochemical surface properties of the particle (PIVs) to be purified which in turn determine the particular chemistry of the chromatographic support utilized, yet another further consideration is being able to maintain infectiousness of the particles being subjected to varying shear forces (e.g., particle shear sensitivity) during purification steps. As a consequence, embodiments of the present invention comprise purification methods utilizing hollow-fiber Tangential Flow Filtration (TFF) system(s) as opposed to a flat sheet system(s), such as TFF systems, provided by, but not limited to, Spectrum Labs, Rancho Dominguez, Calif. TFF filtration (also referred to as Cross Flow Filtration CFF) is well known to those of skill in the art and equipment and protocols for its implementation in a wide range of situations are commercially available from a variety of manufacturers including, but not limited to, the Pall Corporation, Port Washington N.Y. (www.pall.com) and Spectrum Labs, Rancho Dominguez, Calif. Generally, TFF involves the recirculation of the retentate across the surface of the membrane. This gentle cross flow feed minimizes membrane fouling, maintains a high filtration rate and provides high product recovery.

The methods of the present invention may be implemented with a flat sheet system or hollow-fiber systems as exemplified herein. At the laboratory scale many flat-sheet (dead-end filtration) membrane modules contain a turbulence-generating screen to minimize formation of a gel layer during fluid flux. In contrast, the open-channel architecture and cross-flow filtration of the hollow fiber TFF/CFF systems have been shown to be superior for purification of infectious viruses. In certain embodiments, the hollow fiber MWCO is from about 50- to 100- to 500-kDa (or greater). Preferred embodiments of the flavivirus purification methods of the present invention optimized for REPLIVAX® PIV processing utilize hollow fiber TFF in the MWCO range of about 500 kDa. The inventors have found that a MWCO range of about 500 kDa does not reduce the effectiveness of virus retention and allows for a much greater degree of purification during the TFF step.

In some embodiments, particularly those sized for large scale (e.g., commercial) production flat sheet systems are preferred especially where such systems are optionally provided with a means (e.g., an open flow channel) to prevent excessive shear forces on the enveloped (e.g., flavivirus) viral particles.

The present invention describes downstream methods and processes for preparation of highly purified (e.g., in the range from about 99.99 to 99.90, 99.00, 98.00, 95.00, 90.00, 80.00, 70.00, to about 50.00%, and points in between), high-titer flavivirus particles. In preferred embodiments the purified flaviviruses comprise REPLIVAX® single-component pseudoinfectious virus (PIV) particles. The invention further provides a method for the purification and the preparation of purified preparations of flavivirus particles, in particular, where said flavivirus particles are recombinant REPLIVAX® or recombinant CHIMERIVAX® particles, vectors, or constructs.

In one embodiment of the invention, more fully described in the Examples, a method was applied to the purification of recombinant REPLIVAX® West Nile ΔC-prM-E construct(s). Following propagation of REPLIVAX® on the appropriate complementing cell line, it is desirable to purify the virus from the cellular material and the cell culture media components before further use. The REPLIVAX® purification process is a multi-step procedure resulting in pure, high titer infectious REPLIVAX® virions, and non-infectious VLPs. Briefly, at 48-72 hours post infection (hpi), depending upon the infection kinetics for the particular REPLIVAX® vaccine candidate to be purified, the REPLIVAX® containing serum free media is decanted from the monolayer of infected cells and clarified by centrifugation at 2,000×g, for from 10-, to about 20 min at 4° C. Further clarification, for removal of cell debris and other particulate matter, can be performed by filtration (0.8 μm, 25 mm, SUPOR® polyethersulfone (PES) membrane (Pall Corp., PN 4618) prior to ultrafiltration and diafiltration (UF/DF) by hollow fiber tangential flow filtration (TFF) with a module of molecular weight cutoff 500 kDa (e.g. 85 cm², Polysulfone-, Spectrum Labs, PN X2-5002-200-02P). Further concentration and purification is achieved by bind-and-elute anion exchange chromatography using low-shear convective interaction media (CIM® Q, BIA Separations, PN 210.5113) with a quarternary amine functional group. Finally, buffer exchange into an appropriate buffer for cryopreservation is achieved by dialysis. Titers of 1E+9 were achieved.

Ultrafiltration and chromatography are commonly used for downstream processing of cell culture derived virus particles. (Wolff M W, and Reichl U., Expert Rev Vaccines, 2011, 10:1451-1475). Chromatography can in some cases result in loss of infectivity or distortion of virus particles. The high recovery of infectious virus described herein demonstrates that the convective flow of the anion exchange monolithic column(s) (e.g., CIM® Q disk(s)) and the low shear of the hollow-fiber TFF module(s) are gentle enough to preserve infectivity. Thus, the present invention provides novel methods combining, at least, specific chemistries and physical properties of the various media, the order of process steps, and other physical parameters that are optimized for purification and concentration of enveloped viruses, preferably, flaviviruses, and more preferably, REPLIVAX® PIVs, without loss of corresponding infectivity. The methods of the present invention can be easily scaled and therefore applied to downstream manufacturing of clinical preparations and commercial size lots.

In certain embodiments of the invention, where the virus particles to be purified are of particular size to make sterile filtration of the material difficult (i.e. greater than about 200 nm) and where the final material is desired to be sterile, the processes are further performed under sterile conditions and/or with additional sterilizing steps.

The enveloped (e.g., flavivirus) viral particles purified according to the present invention can be formulated according to known methods of preparing pharmaceutically useful compositions. The compositions of the invention may be formulated for administration by manners known in the art acceptable for administration to a mammalian subject, preferably a human. In particular delivery systems may be formulated for intramuscular, intradermal, mucosal, subcutaneous, intravenous, injectable depot type devices or topical administration. When the delivery system is formulated as a solution or suspension, the delivery system is in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized (micropelleted), the lyophilized preparation being combined with a sterile solution prior to administration.

The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

In particular, such pharmaceutical preparations may be administered to mammalian subjects to induce an immune response in the mammalian subject. The intensity of such immune response may be modulated by dosage to range from a minimal response for diagnostic applications (e.g. skin testing for allergies) to a durable protective immune response (immunization) against challenge.

In order to enhance the immune response to the viral particle, such pharmaceutical preparations may optionally include adjuvants. Examples of adjuvants include aluminum salts (e.g. potassium aluminum sulfate, alum, aluminum phosphate, aluminum hydroxyphosphate, aluminum hydroxide), 3D-MPL, oil-in-water emulsions including but not limited to AS03, AF03, AF04, MF-59, and QS21.

The invention further provides pharmaceutically acceptable dosage forms of one or more enveloped viral vector (e.g., REPLIVAX® or CHIMERIVAX® PIVs) produced in cell culture (e.g., mammalian cell culture) wherein the residual host cell DNA in said composition is less than 10 ng host cell DNA per dose, one dose being defined as 500 μL of 2E+7 PFU/mL. Typical mammalian cell hosts for enveloped viruses are well known to those of skill in the art and are readily available from public and private depositories. Particularly useful for the production of viruses exemplified here for purposes of the present invention include the Vero, HEK293, MDK, A549, EB66, CHO and PERC.6

### EXAMPLES

The following examples are to be considered illustrative and not limiting on the scope of the invention described above.

### Example 1

### 1.1 Cells, Virus Seed, and Cell Culture Media

Two of the three REPLIVAX® constructs used herein have been described previously. The RVWNΔC and RVWNΔprM-E viruses, both constructed from the WN NY99 strain each constitute a single-component REPLIVAX® variant. (Rumyantsev A A, et al., Virology, 2010, 396:329-338; Mason P W, et al., Virology, 2006, 351:432-443; and Widman D G, et al., Vaccine, 2008, 26:2762-2771). Additionally, a prototype virus where the F gene from RSV was inserted in place of the prM-E deletion in RVWNΔprME was constructed to evaluate delivery of foreign genes (RVWNΔprME/RSV F; see, e.g., WO 2010/107847). All viruses were propagated on complementing packaging cell lines, which supply the deleted gene(s) in trans (Mason P W, et al., and Widman D G, et al.). Briefly, BHK cells expressing either the WN virus specific C or C-prM-E genes with the puromycin N-acetyl-transferase (PAC) gene expressed in Venezuelan equine encephalitis virus replicons were maintained at 37° C., 5% CO₂ in α-MEM (Life Technologies, Carlsbad, Calif.) supplemented with 5% FBS (HyClone, Waltham, Mass.), vitamins, non-essential amino acids, lx antibiotic/antimycotic mixture and 10 μg/mL puromycin (InVivoGen, San Diego, Calif.). For titration, Vero cells which were originally obtained from the American Type Culture Collection (ATCC, Manassas, Va.) were maintained in MEM (Life Technologies) supplemented with 10% FBS, L-glutamine and 1× antibiotic/antimycotic mixture at 37° C. and 5% CO₂.

### 1.2 REPLIVAX® Upstream Production

BHK helper cells were grown to confluence in T-225 flasks. Cells were then infected at a multiplicity of infection (MOI) of 0.1-1.0 for 1 h at 37° C., 5.0% CO₂ in puromycin-containing growth medium supplemented with 2% FBS. After 1 h the virus-adsorbed cells were overlaid with growth medium containing 5% FBS. At 72 hours post-infection (hpi) the media were harvested and processed as described below.

### 1.3 Centrifugal Concentration of REPLIVAX®

Prior to concentration of REPLIVAX® PIVs, the cell culture supernatant was clarified by centrifugation for 20 min at 4° C. and 2,000×g. The PIVs were then concentrated in a Centricon Plus-70 centrifugal filter unit as per the manufacturers instructions (EMD Millipore, Bedford, Mass.). Concentrated virus was diluted 1:1 with 20% sorbitol in MEM and stored at −80° C.

### 1.4 REPLIVAX® Purification by TFF and Chromatography

The supernatant of REPLIVAX® infected packaging cells was clarified by centrifugation for 20 min. at 2,000×g followed by filtration with a 0.8 μm low protein binding SUPOR® membrane Polyethersulfone (PES) syringe filter (Pall Corporation, Port Washington, N.Y.). Initially, chromatographic separation was performed directly on the cell culture supernatant, though after analysis of the purity of elution fractions it was determined that a hollow fiber tangential flow filtration (TFF) step should preferably precede chromatography for initial purification and concentration of REPLIVAX® PIVs. Post column chromatography, the peak elution fractions were pooled and dialyzed against low salt column equilibration buffer (Buffer A-described below) containing 20% sucrose prior to flash freezing on dry ice/ethanol and storage at −80° C.

During the initial chromatography media screen the following affinity and anion exchange chromatographic resins were tested for their ability to bind and elute infectious REPLIVAX® PIVs: HI-TRAP™ HEPARIN HP (GE Healthcare, Piscataway, N.J.), CELLUFINE® Sulfate (CHISSO Corporation, Tokyo, Japan), HI-TRAP™ CAPTO™ Q (GE Healthcare) and CONVECTION INTERACTION MEDIA (CIM)® Q (BIASeparations, Villach, Austria). All chromatographic separations were performed on an ÄKTA™ purifier automated fast protein liquid chromatography (FPLC) system (GE Healthcare, Sugar Notch, Pa.). Bound REPLIVAX® virions were eluted from the chromatography resin by application of a linear gradient of sodium chloride over 20-30 column volumes (CV) depending on the resin being tested. Chromatography buffers consisted of 50 mM potassium glutamate, 10 mM L-histidine and 10% sucrose, pH 7.5 (“Base Buffer”). The Base Buffer was supplemented with 100 mM NaCl to make the low salt chromatography buffer (“Buffer A”). The Base Buffer was supplemented with 2 M NaCl to make the high salt elution buffer (“Buffer B”). The chromatography resin which was chosen, on the basis of yield of infectious virus particles, was the convective flow monolithic anion exchanger CIM® Q. A two-step (Step 1=35% Buffer B, Step 2=100% Buffer B) elution was used for preparation of pure, high titer, infectious REPLIVAX® PIVs.

The hollow-fiber TFF step prior to column chromatography was performed using 100 or 500 kDa MWCO, 85 cm², polysulfone hollow fiber TFF module (Spectrum Laboratories, Rancho Dominguez, Calif.) on a Kros-Flo® Research II system (Spectrum Laboratories). In order to minimize shear, a low flow rate was utilized (130 mL/min, which equates to a shear rate of 4,000 s⁻¹). The clarified cell culture supernatant was concentrated 2-6-fold by volume (to slightly less than 50 mL) and then diafiltered against 5×50 mL of chromatography Buffer A. The contaminating host cell protein-containing permeate was retained for analysis as 50-150 mL fractions. The transmembrane pressure (TMP) was kept below 4 psi throughout the diafiltration process to minimize formation of a gel layer, which could impede fluid flux. PIV recovery was assessed by titrating samples in Vero cells as described below.

### 1.5 Titration of REPLIVAX®

Infectivity of REPLIVAX® was assessed by titration of samples on Vero cells by immunofocus assay (IFA) as described in Rumyantsev A A, et al. (Rumyantsev A A, et al., Vaccine, 2011, 29:5184-5194). Samples were serially diluted into MEM supplemented with 2% FBS, 2 mM glutamine and 1× antibiotic/antimycotic (Life Technologies, Carlsbad Calif.) and the virus suspension was plated onto Vero cells in 96 well tissue culture plates. Each virus dilution was assayed in quadruplicate. Infection was allowed to proceed 1 h at 37° C. in a 5% CO₂ humidified incubator with gentle rocking every 15-30 min. After the viral adsorption period, the samples were overlaid with 0.1 mL per well of the diluent described above, rocked to mix the overlay and the inoculum, and incubated 24-36 hours. After the incubation period, the cells were fixed and permeabilized with methanol. Individually-infected cells were visualized by immunostaining with mouse anti-WN hyperimmune ascitic fluid (HIAF) followed by goat anti-mouse IgG-Fc HRP conjugated secondary antibodies (Thermo Fisher Scientific/Pierce, Waltham Mass.). Infected cells were visualized by colorimetric development with 0.5 mg/mL 3,3′-diaminobenzidine tetrahydrochloride hydrate (DAB) (Sigma, Saint Louis, Mo.) in 1×PBS with 0.015% H₂O₂ (Sigma). Titers were determined by counting individual stained cells and are expressed in focus forming units (FFU/ml).

### 1.6 SDS-PAGE and Western Blotting

Concentrated or chromatography-purified REPLIVAX® PIV preparations (either 1×10⁶ or 4×10⁶ focus forming units (FFU)/lane, as indicated) were resolved by 4-12% SDS-PAGE (NuPAGE, Bis-Tris, Life Technologies) after heating of the samples 5 min at 95° C. in Laemmli SDS sample loading buffer containing β-mercaptoethanol (Boston BioProducts, Ashland, Mass.). The poylacrylamide gels were either stained with SimplyBlue™ SafeStain (Life Technologies) or transferred to a nitrocellulose membrane using a dry protein transfer on the iBlot transfer apparatus (Life Technologies). The membranes were probed either for WN E or RSV F proteins using a mouse monoclonal anti-WN 7H2 (BioReliance Corporation, Rockville, Md.) or anti-RSV F mouse monoclonal, respectively. Membranes were incubated with an alkaline phosphatase-labeled anti-mouse IgG secondary antibody (Southern Biotech, Birmingham, Ala.) and proteins were visualized using the SIGMAFAST™ BCIP®/NBT (Sigma) chromogenic reagent.

### Example 2

### 2.1 Chromatography Media Screen (RVWNΔC & RVWNΔprM-E)

Initial chromatography resin screening was undertaken on clarified, serum-free cell culture supernatant containing prototype RepilVax®-WN PIVs RVWNΔC or RVWNΔprM-E. The starting titer for chromatographic separation with the RVWNΔC PIV was about 3-5×10⁵ FFU/mL whereas for the RVWNΔprM-E PIV the titer of the starting material was about 4×10⁴ FFU/mL (Table 1, column 2; total FFU Load has been adjusted for volume). Recoveries presented as a % of the total FFU loaded per column are presented in Table 1.

TABLE 1

| | RVWN | Recovery (%)^{a} | | | |
|---|---|---|---|---|---|
| Resin | Construct | Load (FFU) | FT^{b} | Elution | Total |
| MUSTANG ® Q | ΔC | 1.4 × 10⁷ | 19 | 36 | 55 |
| CAPTO ™ Q | ΔC | 2.3 × 10⁶ | 65 | 37 | 102 |
| CIM ® Q | ΔC | 2.3 × 10⁶ | 6 | 72 | 78 |
| CIM ® Q | ΔprM-E | 4.3 × 10⁵ | 18 | 50 | 68 |
| CELLUFINE ® Sulfate | ΔprM-E | 4.3 × 10⁵ | 11 | 16 | 27 |
| HI-TRAP ™ Heparin HP | ΔC | 2.3 × 10⁶ | 33 | 46 | 79 |

Virus yield from small-scale screening of anion exchange and affinity capture reagents. REPLIVAX ® West Nile PIVs were eluted from the chromatographic supports with a 0-100% Buffer B linear gradient.
^{a}Recovery is presented as a % of the total titer (FFU) loaded onto the column;
^{b}FT (flow through).

Initially, the MUSTANG® Q anion exchange membrane (Pall Corp., Port Washington, N.Y.) (Table 1, row 1) was tested as a bind-and-elute chromatographic support, since it had been shown previously to be appropriate for purification of an enveloped virus vaccine candidate. Additional supports were tested to improve upon the recovery of virus eluted from the column. Anion exchange resins CIM® Q and Capto™ Q (which was developed with a long linker arm to tether the functional group to the chromatography bead, specifically for purification of large molecules) as well the sulfated affinity resins HI-TRAP™ Heparin HP and CELLUFINE® Sulfate were all tested for the ability to bind and elute infectious virus. Ultimately, Convective Interaction Medium (media) (CIM® Q) consistently provided the best recovery of infectious material (50%-72%) after elution from the chromatographic support. SDS-PAGE and Western blot analysis of the elution fractions from the chromatography runs presented in Table 1 shows that the material eluting from the chromatographic support contained a significant amount of residual non-viral protein. Due to the level of impurities, it was determined that a two-step purification process would be advantageous to obtaining a more pure virus preparation.

### 2.2 Tangential Flow Filtration for Ultrafiltration/Diafiltration (UF/DF) of REPLIVAX® (RVWNΔC & RVWNΔprM-E/RSV F)

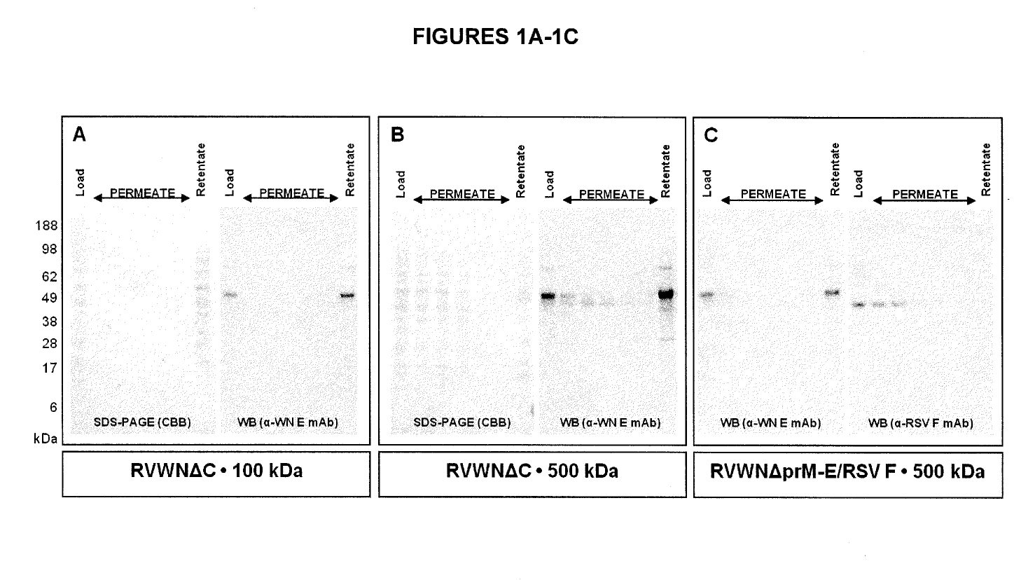

Since the initially purified REPLIVAX® PIVs did not have the purity profile that was hoped for, TFF was explored as a purification step prior to CIM Q chromatography. Previous work has shown that hollow-fiber TFF provides better virus recovery of an enveloped virus vaccine candidate when a 100 kDa MWCO module is used. In an initial experiment (FIG. 1A), the RVWNΔC PIV was concentrated 2-fold by volume and diafiltered against chromatography Buffer A. Although the overall recovery of infectious virus was high (about 80%) no contaminating host cell proteins appeared to be flushed into the permeate during the diafiltration process (FIG. 1A). In contrast, when a 500 kDa MWCO hollow fiber module was used, non-viral proteins were flushed into the permeate, while most of the virus was retained (FIG. 1B) and concentrated (6 fold by volume). Notably, the recovery of infectious virus using the 500 kDa MWCO TFF cassette was the same (about 80%) as with the 100 kDa MWCO module, indicating TFF as a first step in the purification process not only concentrates virus with minimal loss but also partially purifies.

During upstream production of REPLIVAX® PIV vectors expressing a foreign gene (as with RVWNΔprM-E/RSV F), the foreign protein is expressed in packaging cells. During pre-clinical testing, it is preferred that the purified virus is devoid (or nearly devoid) of the foreign protein, e.g., in order to evaluate immunogenicity of the foreign protein synthesized de novo. FIG. 1C shows the presence of WN E protein (left) as well as the RSV F protein (right) in the cell supernatant prior to concentration by TFF module (lanes “load”). The viral envelope protein (PIV particles) is retained throughout the diafiltration process whereas the soluble RSV F protein is washed away. The concentration factor was 2.5-fold by volume and recovery of infectious PIV was >75%.

### 2.3 Bind-and-Elute Chromatography of REPLIVAX® (RVWNΔC & RVWNΔprM-E/RSV F)

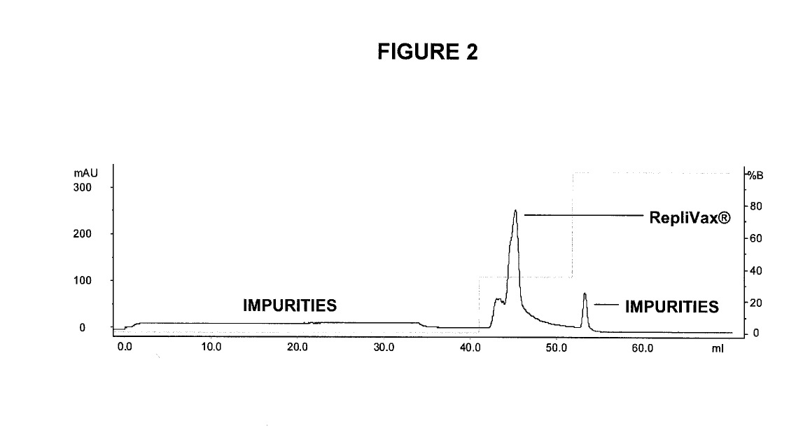

Partially processed REPLIVAX® PIVs (TFF Retentate) in chromatography Buffer A were immediately loaded onto a laboratory scale (0.35-0.7 mL) CIM Q monolith disk for chromatographic separation. FIG. 2 depicts a representative chromatographic profile for binding and elution of RVWNΔC from the CIM Q monolith. The elution profile for all prototype REPLIVAX® samples was identical. The bound REPLIVAX® containing material was eluted from the column at 35% Buffer B (900 mM NaCl). In all cases, the recovery of purified infectious PIVs at this scale (30-60%) was lower than was expected from the preliminary screens (50-70%) suggesting the column capacity had been exceeded. Indeed, 40-70% of the infectious titer did pass through the column in the flowthrough fraction. Under the conditions described here, the capacity of the CIM Q monolith for REPLIVAX® PIVs is about 4.5×10⁹ FFU per mL of monolith bed volume.

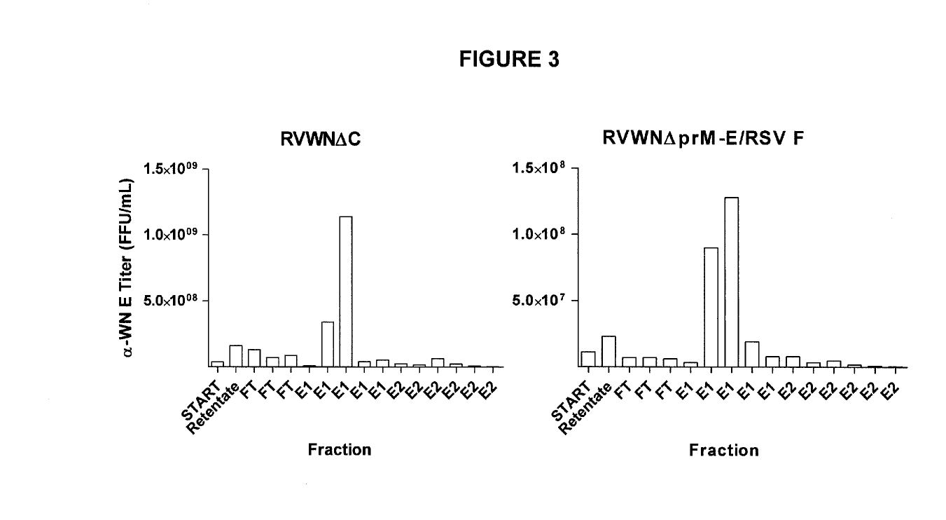

The amount of infectious RVWNΔC and RVWNΔprME/RSVF PIVs present during the purification scheme was assessed and is depicted in FIG. 3. In both cases, the titer of the peak elution fraction was 1-2 orders of magnitude higher than that of the starting material (titers went from 3.7×10⁷ to 1.2×10⁹ FFU/mL for the RVWNΔC PIV and from 2.7×10⁷ to 2.2×10⁸ FFU/mL for the RVVVNAprM-E/RSV F PIV), which corresponded to a concentration factor (by volume) of 100-fold and 25-fold, respectively. In some embodiments, even higher titers are anticipated when the specified (e.g., column capacities) purification apparatus and systems are implemented and specifically matched to purification process goals.

### 2.4 Purity and Recovery of REPLIVAX® (RVWNΔC & RVWNΔprM-E/RSV F)

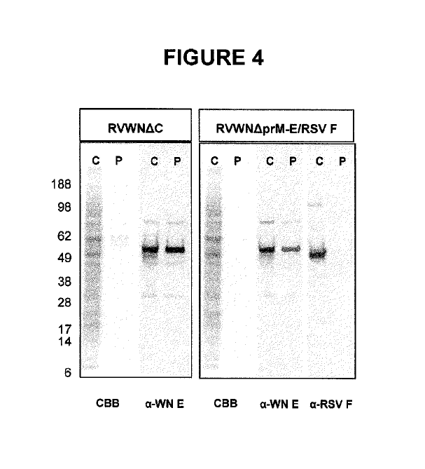

SDS-PAGE analysis of REPLIVAX® PIVs prepared by centrifugal (CENTRICON®, EMD Millipore, Billerica, Mass.) concentration showed that the concentrated preparation contained a smear of contaminating non-viral proteins (FIG. 4, Coomassie Brilliant Blue [CBB] stained SDS-PAGE, Sample C). In contrast, chromatography-purified material was nearly devoid of contaminating proteins (FIG. 4, CBB stained SDS-PAGE, Sample P; the samples were normalized for the same FFU loaded per lane). The amount of the WN E protein detected in these preparations was similar by Western blot (FIG. 4, α-WN E Western blot). Additionally, soluble RSV F protein expressed during production of RVWNΔprM-E/RSV F PIV was efficiently removed during the purification procedure, while it remained present in the centrifugal concentrated preparation (FIG. 4, α-RSV F Western blot).

1. A method for the purification of a flavivirus viral particle from a host cell culture comprising the steps of:
a. recovering flavivirus viral particles from a host cell culture;
b. subjecting the flavivirus viral particles obtained from step (a) to tangential flow filtration;
c. applying the retentate from the tangential flow filtration step to an anion exchange chromatography resin;
d. eluting flavivirus viral particles from the anion exchange chromatography resin; and
e. recovering purified flavivirus viral particles.

2. The method of claim 1 wherein the flavivirus viral particles are recombinant flavivirus viral particles.

3. The method of claim 2 wherein the flavivirus viral particles comprise replication defective pseudoinfectious virus particles or chimeric flaviviruses comprising capsid and non-structural sequences of a yellow fever virus and pre-membrane and envelope sequences of a second, different flavivirus.

4. (canceled)

5. The method of claim 1 wherein the flavivirus viral particles comprise flaviviruses transmitted via an arthropod vector.

6. The method of claim 5 wherein the flaviviruses are selected from the group consisting of yellow fever viruses, Japanese encephalitis viruses, dengue viruses, West Nile viruses, and tick-borne encephalitis viruses.

7. (canceled)

8. The method of claim 6 wherein the yellow fever viruses comprise YF-17D virus.

9. The method of claim 1 wherein the host cell culture comprises mammalian cell culture.

10. The method of claim 1 wherein the tangential flow filtration comprises filtration through one or more hollow fiber TFF cassettes or through a flat sheet media.

11. The method of claim 10 wherein the one or more hollow fiber TFF cassettes comprises a media selected from the following polysulfone, polyethersulfone, modified polyethersulfone or mixed cellulose ester medias.

12-13. (canceled)

14. The method of claim 1 wherein the anion exchange chromatography resin comprises immobilized positively charged moieties or one or more anionic exchange monolithic columns.

15. The method of claim 14 wherein the immobilized positively charged moieties comprise quaternary amino groups or diethylaminoethane groups.

16-17. (canceled)

18. The method of claim 14 wherein the one or more anionic exchange monolithic columns comprises low-shear convective interaction media with a quaternary amine functional group.

19. The method of claim 1 wherein the recovering of flavivirus viral particles from the host cells comprises decanting medium from the host cell culture.

20. A pharmaceutically acceptable dosage form of flavivirus viral particles produced in a host cell culture, said flavivirus viral particles isolated by a method comprising the steps of:
a. recovering flavivirus viral particles from a host cell culture;
b. subjecting the flavivirus viral particles solution obtained from step (a) to tangential flow filtration;
c. applying the retentate from the tangential flow filtration step to an anion exchange chromatography resin;
d. eluting flavivirus viral particles from the anion exchange column;
e. recovering purified flavivirus viral particles; and
f. suspending purified flavivirus viral particles in a pharmaceutically acceptable carrier.

21. The pharmaceutically acceptable dosage form of claim 20 wherein the flavivirus viral particles are recombinant flavivirus viral particles.

22. The pharmaceutically acceptable dosage form of claim 21 wherein the flavivirus viral particles comprise replication defective pseudoinfectious virus particles or chimeric flaviviruses comprising capsid and non-structural sequences of a yellow fever virus and pre-membrane and envelope sequences of a second, different flavivirus.

23. (canceled)

24. The pharmaceutically acceptable dosage form of claim 21 wherein the flavivirus viral particle are flaviviruses transmitted via an arthropod vector.

25. The pharmaceutically acceptable dosage form of claim 24 wherein the flaviviruses are selected from the group consisting of yellow fever viruses, Japanese encephalitis viruses, dengue viruses, West Nile viruses, and tick-borne encephalitis viruses.

26. (canceled)

27. The pharmaceutically acceptable dosage form of claim 25 wherein the yellow fever viruses comprise YF-17D virus.

28. The pharmaceutically acceptable dosage form of claim 20 wherein the host cell culture comprises mammalian cell culture.

29. The pharmaceutically acceptable dosage form of claim 20 wherein the tangential flow filtration comprises filtration through one or more hollow fiber TFF cassettes or through a flat sheet media.

30. The pharmaceutically acceptable dosage form of claim 29 wherein the one or more hollow fiber TFF cassettes comprises a media selected from the following polysulfone, polyethersulfone, modified polyethersulfone or mixed cellulose ester medias.

31-32. (canceled)

33. The pharmaceutically acceptable dosage form of claim 20 wherein the anion exchange chromatography resin comprises immobilized positively charged moieties or one or more anionic exchange monolithic columns.

34. The pharmaceutically acceptable dosage form of claim 33 wherein the immobilized positively charged moieties comprise quaternary amino groups or diethylaminoethane groups.

35-36. (canceled)

37. The pharmaceutically acceptable dosage form of claim 33 wherein the one or more anionic exchange monolithic columns comprises low-shear convective interaction media with a quaternary amine functional group.

38. The pharmaceutically acceptable dosage form of claim 20 wherein the quantity of host cell DNA in said composition is less than 10 ng host cell DNA per dose, one dose being defined as 500 μL of 2E+7 PFU/mL.
